(19) United States
(12) Patent Application Publication
(10) Pub. No.: US 2022/0117545 A1
(43) Pub. Date: Apr. 21, 2022

(54) SYSTEMS AND METHODS FOR DIFFERENTIALLY DETECTING SUBTLE ONSET OF INFECTION, ACTIVITY LEVEL, AND PERIWOUND HYDRATION FOR ENHANCED WOUND MONITORING

(71) Applicant: KCI LICENSING, INC., San Antonio, TX (US)

(72) Inventors: Justin A. LONG, Bournemouth (GB); Christopher B. LOCKE, Bournemouth (GB)

(21) Appl. No.: 17/425,171

(22) PCT Filed: Feb. 5, 2020
(86) PCT No.: PCT/US2020/016757

§ 371 Date: Jul. 22, 2021

Related U.S. Application Data
(60) Provisional application No. 62/806,472, filed on Feb. 15, 2019.

(51) Int. Cl. A61B 5/00; A61B 5/01; A61B 5/145; A61B 5/11; G16H 20/30; G16H 40/67; A61M 1/00

(52) U.S. Cl. CPC A61B 5/445; A61B 5/01; A61B 5/14539; A61B 5/11; A61B 5/4836; G16H 20/30; A61M 2205/3344; G16H 40/67; A61B 5/443; A61M 1/90; A61B 5/4842; A61M 2205/3303; A61B 5/0024

(57) ABSTRACT

A wound therapy system includes a first sensor assembly, a second sensor assembly, and a processing circuit. The first sensor assembly is configured to be located at a wound treatment site on a patients body and to record physical characteristics representative of periwound tissue at the wound treatment site. The second sensor assembly is configured to be located at a healthy tissue site on the patients body contralateral to the wound treatment site and to record one or more physical characteristics representative of healthy tissue at the healthy tissue site. The physical characteristics representative of the healthy tissue are recorded concurrently with the physical characteristics representative of the periwound tissue. The processing circuit is configured to receive the one or more physical characteristics representative of the periwound tissue from the first sensor assembly at the wound treatment site, receive the one or more physical characteristics representative of the healthy tissue from the second sensor assembly at the healthy tissue site, and determine a healing progression of the wound based on a difference between the one or more physical characteristics representative of the periwound tissue and the one or more physical characteristics representative of the healthy tissue.

### CROSS-REFERENCE TO RELATED APPLICATIONS

This application claims the benefit of priority to U.S. Provisional Application No. 62/806,472, filed on Feb. 15, 2019, which is incorporated herein by reference in its entirety.

### BACKGROUND

The present disclosure relates generally to a wound therapy system, and more particularly, a wound therapy system that can monitor physiological characteristics of a wound and/or a wound treatment site.

Wounds (e.g., surgical wounds, ulcers, etc.) can become infected. Infected wounds can have different physiological characteristics than uninfected wounds and healthy, unwounded tissue. Exemplary physiological characteristics are different between infected and uninfected tissue include temperature, color, edema, pain, and discharge than healthy tissue. Accordingly, it can be advantageous to monitor the physiological characteristics of wounded tissue to identify infected wounds.

### SUMMARY

One implementation of the present disclosure is a wound therapy system. The wound therapy system includes a first sensor assembly, a second sensor assembly, and a processing circuit. The first sensor assembly is configured to be located at a wound treatment site on a patient's body and to record one or more physical characteristics representative of periwound tissue at the wound treatment site. The second sensor assembly is configured to be located at a healthy tissue site on the patient's body contralateral the wound treatment site and to record one or more physical characteristics representative of healthy tissue at the healthy tissue site. The physical characteristics representative of the healthy tissue are recorded concurrently with the physical characteristics representative of the periwound tissue. The processing circuit is configured to receive the one or more physical characteristics representative of the periwound tissue from the first sensor assembly at the wound treatment site, receive the one or more physical characteristics representative of the healthy tissue from the second sensor assembly at the healthy tissue site, and determine a healing progression of the wound based on a difference between the one or more physical characteristics representative of the periwound tissue and the one or more physical characteristics representative of the healthy tissue.

Another implementation of the present disclosure is a method for monitoring wound healing. The method includes recording one or more physical characteristics representative of periwound tissue at a wound treatment site on a patient's body using a first sensor assembly located at the wound treatment site. The method includes recording one or more physical characteristics representative of healthy tissue at a healthy tissue site on the patient's body contralateral the wound treatment site using a second sensor assembly located at a healthy tissue site. The physical characteristics representative of the healthy tissue are recorded concurrently with the physical characteristics representative of the periwound tissue. The method includes transmitting the one or more physical characteristics representative of the periwound tissue from the first sensor assembly to a processing circuit. The method includes transmitting the one or more physical characteristics representative of the healthy tissue from the second sensor assembly to the processing circuit. The method includes determining, by the processing circuit, a healing progression of the wound based on a difference between the one or more physical characteristics representative of the periwound tissue and the one or more physical characteristics representative of the healthy tissue.

Those skilled in the art will appreciate that the summary is illustrative only and is not intended to be in any way limiting. Other aspects, inventive features, and advantages of the devices and/or processes described herein, as defined solely by the claims, will become apparent in the detailed description set forth herein and taken in conjunction with the accompanying drawings.

### BRIEF DESCRIPTION OF THE FIGURES

FIG. 1 is a schematic representation of a wound therapy system including wound sensor assemblies according to an exemplary embodiment.

FIG. 2 is a schematic representation of a wound therapy system including wound sensor assemblies according to another exemplary embodiment.

FIG. 3 is a schematic representation of the wound therapy system of FIG. 1 deployed on a patient according to an exemplary embodiment.

FIG. 4 is a schematic representation of the wound therapy system of FIG. 2 deployed on a patient according to an exemplary embodiment.

FIG. 5 is a schematic representation of a controller of the wound therapy system of FIG. 1 according to an exemplary embodiment.

FIG. 6 is a schematic representation of a controller of the wound therapy system of FIG. 2 according to an exemplary embodiment.

FIG. 7 is a flow diagram of a method for determining a likelihood that a wound treatment site is infected according to an exemplary embodiment.

FIG. 8 is a flow diagram of a method for determining a likelihood that a patient is complying with a wound treatment regimen according to an exemplary embodiment.

### DETAILED DESCRIPTION

### Overview

Referring generally to the FIGURES, a wound therapy system and method for treating and monitoring wound treatment sites on a patient's body is shown, according to various embodiments. More specifically, the wound therapy system and method includes monitoring physiological characteristics representative of the wound treatment site and monitoring physiological characteristics representative of a healthy tissue site contralateral the wound treatment site. The term “contralateral” as used herein refers to a side of the body opposite to that on which a particular condition (e.g., wound) occurs. The physiological characteristics can include one or more of a temperature, moisture, motion, and pH. For example, a patient can have a wounded left arm or shoulder and an unwounded right arm or shoulder. In another example, a patient can have a wounded left hip or leg and an unwounded right hip or leg. In conditions in which a wounded body part has a contralateral non-wounded body part, the physiological characteristics of the wound treatment site and a healthy tissue site contralateral to the wound treatment site comparison site can be monitored by wound sensor assemblies.

The monitoring can include determining a difference between the physiological characteristics representative of the wound treatment site and the contralateral healthy tissue site. Comparing the wound treatment site with the contralateral healthy tissue site can compensate for person-to-person variability in physiological characteristics. The monitoring can include comparing the difference between the physiological data representative of the wound treatment site and the healthy tissue site to a predetermined threshold. In response to the difference exceeding the predetermined threshold, an alert can be sent to a caregiver indicating that the wound treatment site should be examined.

In in some embodiments, a first wound sensor assembly is configured to be positioned at or proximate the wound treatment site to monitor the periwound tissue. In some embodiments, a second wound sensor assembly is configured to be positioned at the healthy tissue site contralateral to the wound treatment site. The wound sensor assemblies can include one or more sensors configured to sense he physiological characteristics representative of the wound treatment site or the healthy tissue site, a communication interface, and a battery. The wound sensor assemblies can be in wired or wireless communication with a wound therapy system or a client device that includes a controller. The controller can receive the physiological characteristic data representative of the wound treatment site from the first wound sensor assembly. The controller can receive the physiological characteristic data representative of the healthy tissue site from the second wound sensor assembly. The controller can determine a difference between the physiological characteristics representative of the wound treatment site and the physiological characteristics representative of the healthy tissue site. The controller can compare the difference to a predetermined threshold. The controller can determine a likelihood that the wound treatment site requires examination by a healthcare professional in response to comparison. For example, in some embodiments, the controller can determine that the wound treatment site is unlikely to require examination from a healthcare professional in response to the comparison indicating that a difference between the physiological characteristics representative of the wound treatment site and the physiological characteristics representative of the healthy tissue site is less than a predetermined threshold. The controller can determine that the wound treatment site likely requires examination by a healthcare professional in response to the comparison indicating that a difference between the physiological characteristics of the wound treatment site and the healthy treatment site exceeds a predetermined threshold. The controller can send a notification to a health care provider or the patient in response to determining that the wound treatment site is likely requires examination by a healthcare professional.

In another embodiment, the controller can determine a likelihood that a patient is complying with a wound therapy regime. In such an embodiment, the controller can compare the physiological characteristics representative of the wound treatment site to the physiological characteristics representative of the healthy tissue site. The controller can determine a likelihood that the patient is complying with the wound therapy regimen based on the comparison of the physiological characteristics representative of the wound treatment site and the healthy tissue site. The controller can send a notification to a health care provider or the patient in response to determining that the patient is likely not complying with wound therapy regime.

Additional features and advantages of the wound therapy system are described in detail below.

### Wound Therapy System

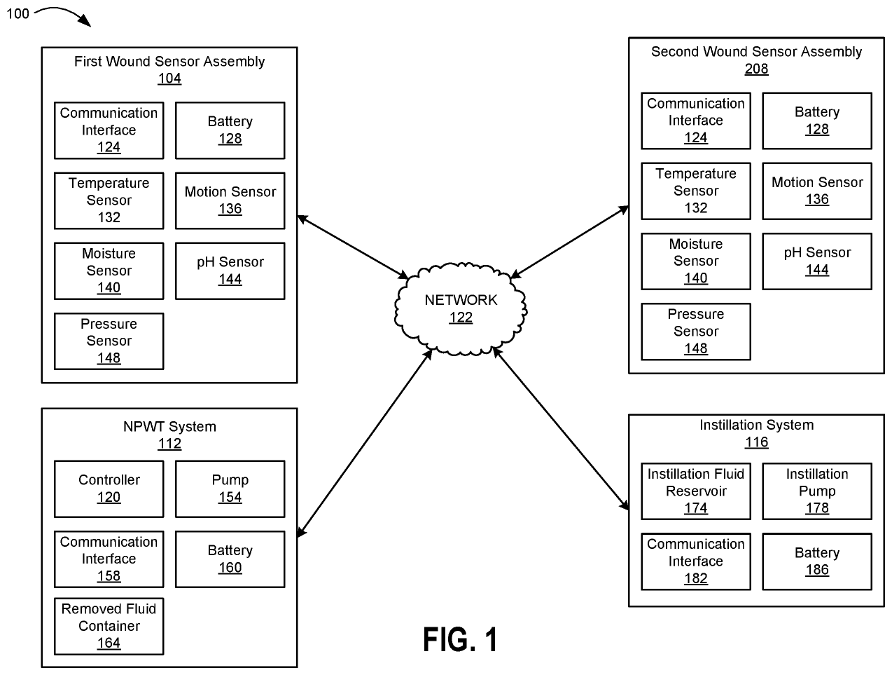

Referring now to FIG. 1 a wound therapy system 100 is shown, according to an exemplary embodiment. The wound therapy system 100 includes a wound treatment site or first wound sensor assembly 104, a healthy tissue site or second wound sensor assembly 108, a negative pressure wound therapy (NPWT) system 112, and an optional instillation system 116. The NPWT system 112 illustrated in FIG. 1 is a powered NPWT system that includes a controller 120. The first wound sensor assembly 104, the second wound sensor assembly 108, the NPWT system 112, and the instillation system 116 can communicate over a network 122 (e.g., a WAN, the Internet, a cellular network, etc.).

The first wound sensor assembly 104 is configured to be secured at or proximate a wound treatment site on a patient. The wound treatment site can include an incision and a periwound portion of a patient's tissue surrounding the incision. In some embodiments, the first wound sensor assembly 104 may be integrated with a wound dressing deployed at the wound treatment site. The wound dressing can include a negative pressure and/or instillation wound dressing used with the NPWT system 112 and/or the instillation system 116. In some embodiments, the first wound sensor assembly 104 is separate from the wound dressing. The second wound sensor assembly 108 is configured to be secured at a healthy tissue site that is contralateral to the wound treatment site. Sensing the physical characteristics of the wound treatment site (e.g., with the first sensor assembly 104) and a healthy tissue site (e.g., with the second sensor assembly 108) that is on the same patient and contralateral to the wound treatment site as described in the present disclosure yields data that may be compared without introducing person-to-person variability and greatly reducing variability based on position along the extremity. Further, comparing the wound treatment site to the contralateral healthy tissue site on the same patient can compensate for person-to-person variability in physiological characteristics because, rather than comparing the physiological characteristics representative of the wound treatment site to a threshold value aggregated across multiple patients, the wound treatment site is compared to the contralateral healthy tissue site on the same patient. This reduction in sources of variability between the physical characteristics sensed at the wound treatment site and the contralateral healthy tissue site increases the likelihood that differences in the physical characteristics are due to one or more conditions at the wound treatment site that require caregiver attention (rather than differences due to other variability factors). Therefore, a difference in the physiological characteristics between a wound treatment site and a contralateral healthy tissue site may indicate that the wound treatment site requires examination by a caregiver.

The first wound sensor assembly 104 is substantially similar to the second wound sensor assembly 108. Accordingly, the only the first wound sensor assembly 104 is described herein. Like parts of the first wound sensor assembly 104 and second wound sensor assembly 108 are illustrated using like numbering. Where sensors and monitoring data with respect to the first wound sensor assembly 104 is discussed below, it is contemplated that the second wound sensor assembly 108 includes similar sensors and monitors similar data representative of the healthy tissue site.

As illustrated in FIG. 1, the first wound sensor assembly 104 includes one or more sensors 132-148, a communication interface 124, and a battery 128. The one or more sensor(s) 132-148 are configured to sense one or more physiological characteristics representative of the periwound tissue at the wound treatment site. For example, the one or more sensor(s) 132-148 can include a temperature sensor 132 configured to sense temperature data, a motion sensor 136 configured to sense motion data, a moisture sensor 140 configured to sense moisture data, a pH sensor 144 configured to sense pH data, and a pressure sensor 148 configured to sense pressure data. The physiological characteristics representative of the wound treatment site can include one or more of a periwound temperature, motion of the extremity including the wound treatment site, periwound moisture, periwound pH, and/or periwound pressure.

The communication interface 124 is configured to receive and transmit data. The communication interface 124 may include wired or wireless communications interfaces (e.g., jacks, antennas, transmitters, receivers, transceivers, wire terminals, etc.) for conducting data communications external systems or devices. In various embodiments, the communications may be direct (e.g., local wired or wireless communications) or via a communications network (e.g., a WAN, the Internet, a cellular network, etc.). For example, the communication interface 124 can include a USB port or an Ethernet card and port for sending and receiving data via an Ethernet-based communications link or network. In another example, communication interface 124 can include a Wi-Fi transceiver for communicating via a wireless communications network or cellular or mobile phone communications transceivers.

The battery 128 is configured to provide power to the one or more sensor(s) 132-148 and the communication interface 124. The battery 128 can be a lithium-ion, nickel-metal hydride, nickel zinc, or another compact or lightweight battery type.

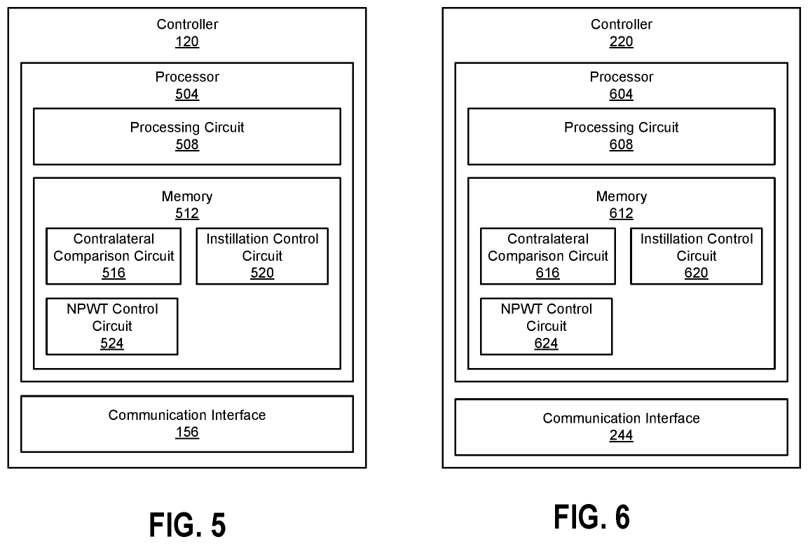

The NPWT system 112 includes the controller 120, a negative pressure source or pump 152, a communication interface 156, a battery 160, and a removed fluid container 164. The communication interface 156 is substantially similar to the communication interface 124. In some embodiments, the communication interface 156 is included in the controller, as illustrated in FIG. 5. The battery 160 can be a lithium-ion, nickel-metal hydride, nickel zinc, or another compact or lightweight battery type. As is discussed in greater detail below, the controller 120 is configured to physiological characteristic data representative of the wound treatment site, physiological characteristic data representative of the healthy tissue site, and to determine a wound healing progression based on a difference between the physiological characteristic data representative of the periwound tissue at the wound treatment site and healthy tissue at the healthy tissue site. The controller 120 is also configured to control the negative pressure source 152 of the NPWT system 112 and a pump 178 of the instillation device 116 as described in greater detail below.

The negative pressure source 152 is in fluid communication with a wound dressing 312, 412 positioned at the wound treatment site. The negative pressure source 152 is configured to apply negative pressure ranging from approximately 50 mmHg-approximately 200 mg to the wound treatment site. The removed fluid container 164 is in fluid communication with the wound dressing 312, 412 to store fluid removed from the incision. Removed fluid can include, for example, wound exudate (e.g., bodily fluids), air, or any other type of fluid which can be removed from the incision 240 during wound treatment. In embodiments in which the wound therapy system 100 includes the instillation device 116, the removed fluid container 164 can collect instillation fluid that has been removed from the wound treatment site.

The instillation device 116 includes an instillation fluid reservoir 174, an instillation pump 178, and a communication interface 182. The communication interface 182 is substantially similar to the communication interface 124. As is described in greater detail below, the instillation device 116 and the controller 120 can communicate over the network 122 via communication interfaces 182, 124. As is discussed in greater detail below, the controller 120 can command the instillation pump 178 provide instillation fluid to the wound treatment site.

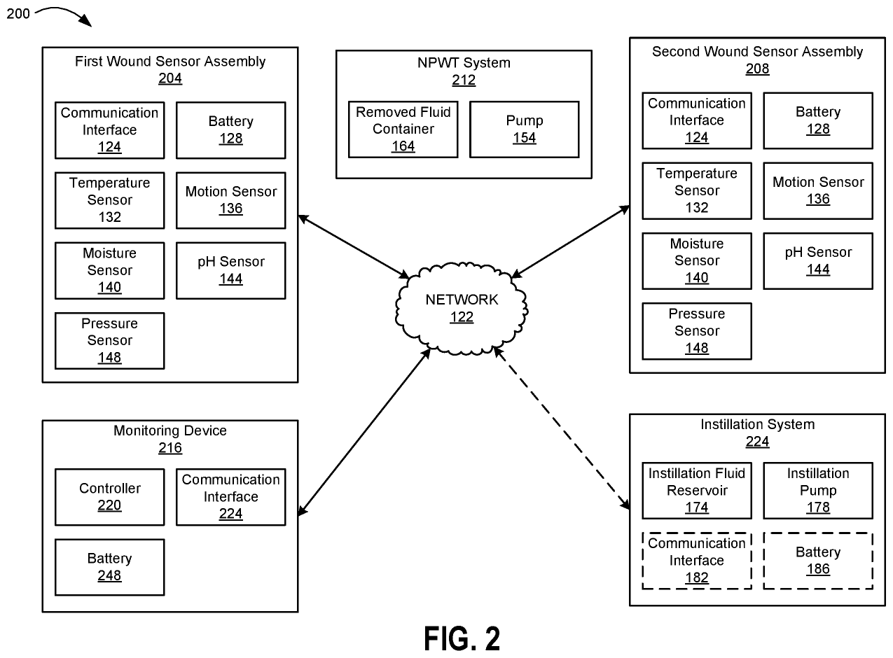

Referring now to FIG. 2, a wound therapy system 200 is shown, according to another exemplary embodiment. The wound therapy system 200 includes a wound treatment site or first wound sensor assembly 204, a healthy tissue site or second wound sensor assembly 208, a NPWT system 212, a monitoring device 216 including a controller 220, and an optional instillation system 224. The NPWT system 212 is a non-powered NPWT system. The optional instillation system 224 may be a powered or anon-powered instillation system. The first wound sensor assembly 204, the second wound sensor assembly 208, the monitoring device 216, and the instillation system 224 can communicate over a network 228. The first wound sensor assembly 204, the second wound sensor assembly 208, the instillation system 224, and the network 228 are substantially the same as the first wound sensor assembly 104, the second wound sensor assembly 108, the instillation system 116, and the network 122 described above with respect to the wound therapy system 100 of FIG. 1 and are not discussed in detail herein.

The NPWT system 212 includes a negative pressure source or pump 232 and a removed fluid container 236. The negative pressure source 232 is in fluid communication with a wound dressing 240 positioned at the wound treatment site. The negative pressure source 232 may be an operator-actuated pump that is configured to apply negative pressure ranging from approximately 50 mmHg-approximately 200 mg to the wound treatment site. The removed fluid container 236 is in fluid communication with the wound dressing 240 to store a removed fluid from the incision. Removed fluid can include, for example, wound exudate (e.g., bodily fluids), air, or any other type of fluid which can be removed from the incision 240 during wound treatment. In embodiments in which the wound therapy system 200 includes the instillation system 224, the removed fluid container 236 can collect instillation fluid that has been removed from the wound treatment site.

The monitoring device 216 includes the controller 220, a communication interface 244, and a battery 248. The controller 220 is in communication with the first wound sensor assembly 204 and the second wound sensor assembly 208 over the network 226. As is discussed in greater detail below, the controller 220 is configured to receive physiological characteristic data representative of periwound tissue and healthy tissue and to determine a wound healing progression based on a difference between the physiological characteristic data of the periwound tissue and healthy tissue physiological characteristics. The communication interface 220 is substantially similar to the communication interface 124. In some embodiments, the communication interface 244 is included in the controller, as illustrated in FIG. 6. The battery 248 can be a lithium-ion, nickel-metal hydride, nickel zinc, or another compact or lightweight battery type. In some embodiments, the monitoring device 216 can be a personal computing device such as a smartphone, a tablet, or a smart watch or other wearable personal computing device.

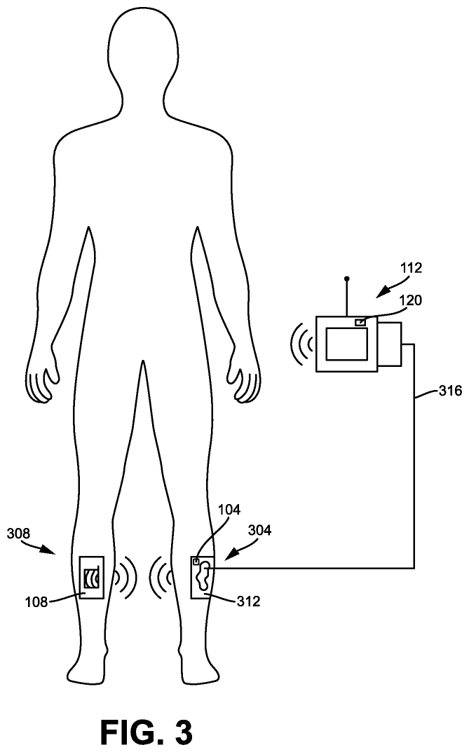

Referring now to FIG. 3, a schematic representative of the wound treatment device 100 deployed on a patient is illustrated, according to an exemplary embodiment. In the exemplary embodiment of FIG. 3, the patient's left leg includes a wound treatment site 304 and the patient's right leg includes a healthy tissue site 308 contralateral to the wound treatment site 304. The wound treatment site 304 is undergoing NPWT provided by the powered NPWT system 112. The wound treatment site 304 includes a NPWT wound dressing 312, which is in fluid communication with the negative pressure source (not shown) of the NPWT system 112 by a negative pressure conduit 316. In the illustrated embodiment, the first wound sensing assembly 104 is integrated with the wound dressing 312. The second wound sensing assembly 108 is secured to the healthy tissue site 308 contralateral to the wound treatment site 304. As indicated in FIG. 3, the first wound sensor assembly 104 and the second wound sensor assembly 108 are wirelessly communicatively coupled to the controller 120 of the NPWT system 112.

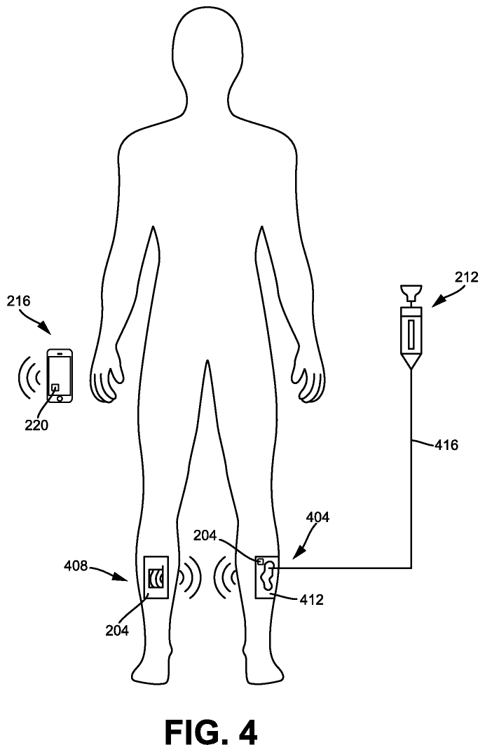

Referring now to FIG. 4, a schematic representative of the wound treatment device 200 deployed on a patient is illustrated, according to an exemplary embodiment. In the exemplary embodiment of FIG. 4, the patient's left leg includes a wound treatment site 404 and the patient's right leg includes a healthy tissue site 408 contralateral to the wound treatment site 404. The wound treatment site 404 is undergoing NPWT provided by the non-powered NPWT system 212. The wound treatment site includes a NPWT wound dressing 412, which is in fluid communication with the negative pressure source (not shown) of the NPWT system 212 by a negative pressure conduit 416. In the illustrated embodiment, the first wound sensing assembly 204 is integrated with the wound dressing 412. The second wound sensing assembly 208 is secured to the healthy tissue site 408 contralateral to the wound treatment site 404. As indicated in FIG. 4, the first wound sensor assembly 204 and the second wound sensor assembly 208 are wirelessly communicatively coupled to the controller 220 of the monitoring device 216.

Referring now to FIG. 5, a block diagram illustrating the controller 120 in greater detail is shown, according to an exemplary embodiment. The controller 120 can be used with various wound dressings and sensors described herein.

The controller 120 includes a processing circuit 504 including a processor 508 and memory 512. The processor 508 may be a general purpose or specific purpose processor, an application specific integrated circuit (ASIC), one or more field programmable gate arrays (FPGAs), a group of processing components, or other suitable processing components. The processor 508 is configured to execute computer code or instructions stored in the memory 512 or received from other computer readable media (e.g., CDROM, network storage, a remote server, etc.).

The memory 512 may include one or more devices (e.g., memory units, memory devices, storage devices, etc.) for storing data and/or computer code for completing and/or facilitating the various processes described in the present disclosure. The memory 512 may include random access memory (RAM), read-only memory (ROM), hard drive storage, temporary storage, non-volatile memory, flash memory, optical memory, or any other suitable memory for storing software objects and/or computer instructions. The memory 512 may include database components, object code components, script components, or any other type of information structure for supporting the various activities and information structures described in the present disclosure. The memory 512 may be communicably connected to the processor 508 via the processing circuit 504 and may include computer code for executing (e.g., by the processor 508) one or more processes described herein. When the processor 508 executes instructions stored in the memory 512, the processor 508 generally configures the controller 120 (and more particularly processing circuit 504) to complete such activities. It will be appreciated that some or all the functions executed by the controller 120 may be executed by a remote computing device, such as a device for use by a clinician that is communicatively coupled the wound sensor assemblies 104, 108, 204, 208, the NPWT device 112, or the monitoring device 216.

The processing circuit 504 includes a contralateral comparison circuit 516 and a NPWT control circuit 520. In embodiments in which the wound therapy system 100 includes the instillation device 116, the processing circuit 504 also includes an instillation control circuit 524.

The contralateral comparison circuit 516 is in communication with the first wound sensor assembly 104 and the second wound sensor assembly 108 over the network 122. The contralateral comparison circuit 516 is configured to receive physiological characteristic data representative of the wound treatment site and the healthy tissue site sensed by the one or more sensors 132-148 of the first wound sensor assembly 104 and the second wound sensor assembly 108, respectively. In some embodiments, the contralateral comparison circuit 516 is configured to alternately or sequentially pair with the first wound sensor assembly 104 to receive information indicative of the physiological characteristics of the wound treatment site and with the second wound sensor assembly 108 to receive information indicative of the physiological characteristics of the healthy tissue site. In other embodiments, the controller 120 can receive data from the first wound sensor assembly 104 and the second wound sensor assembly 108 continuously and distinguish between data received from the first wound sensor assembly 104 and data received by the second wound sensor assembly 108 based on identity information (e.g., IP address, IMEI, etc.) of the first wound sensor assembly 104 and the second wound sensor assembly 108.

The contralateral comparison circuit 516 is configured to determine a difference between the physiological characteristics representative of the wound treatment site and the physiological characteristics representative of the healthy tissue site. The phrase “difference” as recited herein can refer to the value obtained by subtracting the physiological characteristic data representative of the wound treatment site and the healthy tissue site. In some embodiments, the difference can be the absolute value of the value obtained by subtracting the physiological characteristic data representative of the wound treatment site and the healthy tissue site.

The contralateral comparison circuit 516 is configured to compare the difference to a predetermined threshold to determine wound healing progression information. In some embodiments, the wound healing progression information may include a likelihood that the wound treatment site includes one or more situations requiring caregiver attention. Conditions requiring caregiver attention may include conditions such as include infection, overuse of the extremity including the wound treatment site, maceration, and a presence of a wound healing inhibition. In some embodiments, the predetermined threshold can be a value or range of values for which the difference between the physiological characteristics of the wound treatment site and the physiological characteristics of the healthy tissue site indicates that the wound is likely progressing without situations requiring caregiver attention. In some embodiments, the contralateral comparison circuit 516 is configured to determine that the wound treatment site is likely progressing with one or more situations requiring caregiver attention in response to the difference between the physiological characteristics representative of the wound treatment site and the physiological characteristics representative of the healthy tissue site exceeding a predetermined threshold. The contralateral comparison circuit 516 is configured to send a notification to a patient and/or a caregiver based on the comparison indicating a situation requiring caregiver attention. In some embodiments, the notification can include a visual notification, an acoustic notification, a tactile notification (e.g., vibration), a notification message, etc.

In some embodiments, the wound healing progression information can include a likelihood that a patient is complying with a wound therapy regimen. The wound therapy regimen can include motion limitations (e.g., reduced frequency of use, range of motion, etc.), an amount of negative pressure applied to the wound treatment site, a duration of negative pressure applied to the wound treatment site, an amount of instillation fluid applied to the wound treatment site, a dwell time of instillation fluid at the wound treatment site, etc. The predetermined threshold can be a value or range of values for which the difference between the physiological characteristics representative of the wound treatment site and the physiological characteristics representative of the healthy tissue site indicates that a patient is likely complying with a wound therapy regimen. The contralateral comparison circuit 516 is configured to determine that the patient is likely not complying with a wound treatment regimen in response to the difference between the physiological characteristics of the wound treatment site and the physiological characteristic of the healthy tissue site not exceeding a predetermined threshold. For example, a wound treatment regimen may include immobilization or reduced use of the extremity including the wound treatment site. In such embodiments, the difference not exceeding the predetermined threshold may indicate that the patient is overusing the extremity including the wound treatment site. In another example, the wound treatment regimen may include applying negative pressure to the wound treatment site. In such embodiments, the difference not exceeding the predetermined threshold may indicate that the patient is not providing enough negative pressure to the wound treatment site. The contralateral comparison circuit 516 is configured to send a notification to a patient and/or a caregiver based on the comparison indicating likely non-compliance. In some embodiments, the notification can include a visual notification, an acoustic notification, a tactile notification (e.g., vibration), a notification message, etc. The contralateral comparison circuit 516 is configured to determine that the patient is likely complying with a wound treatment regimen in response to the difference between the physiological characteristics of the wound treatment site and the physiological characteristic of the healthy tissue site exceeding a predetermined threshold.

In some embodiments, the contralateral comparison circuit 516 can be configured to save timestamped physiological characteristics data of the wound treatment site, timestamped physiological characteristic data of the healthy tissue site, and/or timestamped differences between the physiological characteristics of the wound treatment site and healthy tissue site to preserve a history of the physiological characteristics. The contralateral comparison circuit 516 can be configured to display the timestamped data to a patient and/or a caregiver over the network to allow the patient and/or the caregiver to visualize data indicative of the wound healing progression of the wound treatment site. The displayed data can include data plots, data trend lines, average data, etc. The data can be displayed on the NPWT system 112 including the controller 120, the mobile device 116 including the controller 220, or sent to other computing devices over the network 122.

In some embodiments, the contralateral comparison circuit 516 can be configured to analyze the timestamped data to analyze the physical characteristics of the wound treatment site over a period of time. For example, the contralateral comparison can determine improvements in the wound treatment site over time. For example, in embodiments in which the physiological characteristic includes temperature, pH, or moisture, a decrease in the difference between the physiological data representative of the wound treatment site and the physiological data representative of the healthy tissue site over time may indicate improvement in a condition of the periwound tissue. In embodiments in which the physiological characteristic includes motion, an increase in a range of motion, frequency of motion, and improvements in gait in the motion data representative of the wound treatment site over time may indicate improvement in a condition of the periwound tissue.

In some embodiments, the physiological characteristic is a temperature and the first wound sensor assembly 104 and the second wound sensor assembly 108 each includes the temperature sensor 132. The contralateral comparison circuit 516 receives temperature data representative of the wound treatment site from the first wound sensor assembly 104 and receives temperature data representative of the healthy tissue site from the second wound sensor assembly 108. The contralateral comparison circuit 516 determines the difference between the temperature of the wound treatment site and the temperature of the healthy tissue. The contralateral comparison circuit 516 compares the temperature difference to a predetermined temperature threshold to determine wound healing progression information. In response to the comparison indicating that the temperature difference is below the predetermined temperature threshold, the contralateral comparison circuit 516 determines that the wound treatment site is likely not infected. In response to the comparison indicating that the temperature difference is above the temperature threshold, the contralateral comparison circuit 516 determines that wound treatment site is likely infected. The contralateral comparison circuit 516 can send a notification to a physician and/or the patient indicating that the wound treatment site is likely infected.

In some embodiments, the physiological characteristic is motion and the first wound sensor assembly 104 and the second wound sensor assembly 108 each includes the motion sensor 136. In some embodiments, the motion sensor 136 can be an accelerometer. The contralateral comparison circuit 516 receives motion data representative of the extremity including wound treatment site from the first wound sensor assembly 104 and receives motion data representative of the extremity including of the healthy tissue site from the second wound sensor assembly 108. The motion data can include information indicative of a gait, information indicative of a frequency of motion, information indicative of a range of motion, and/or information indicative of an amount of motion. The contralateral comparison circuit 516 then determines the difference between the motion data representative of the wound treatment site and the motion data representative of the healthy tissue site. The contralateral comparison circuit 516 then compares the motion difference to a predetermined motion threshold to determine wound healing progression information. In response to the comparison indicating that the difference in motion is above the motion difference threshold, the contralateral comparison circuit 516 determines that the patient is likely complying with a wound treatment regimen. For example, a treatment regimen can include reducing a frequency of motion and/or range of motion of an injured extremity. The contralateral comparison circuit 516 can send a notification to a physician and/or the patient indicating that the patient is not complying with the wound treatment regimen. A difference in frequency of motion and/or range of motion between the extremity including the wound treatment site and the extremity including the healthy tissue site that does not exceed the predetermined threshold can indicate overuse of the extremity including the wound treatment site.

In some embodiments, the physiological characteristic is moisture and the first wound sensor assembly 104 and the second wound sensor assembly 108 each includes the moisture sensor 140. In some embodiments, the moisture sensor 140 is a humidity sensor. The contralateral comparison circuit 516 receives moisture data representative of the wound treatment site from the first wound sensor assembly 104 and motion data representative of the healthy tissue site from the second wound sensor assembly 108. The contralateral comparison circuit 516 then determines the difference between the moisture of the wound treatment site and the moisture of the healthy tissue site. The contralateral comparison circuit 516 then compares the moisture difference to a predetermined moisture threshold to determine wound healing progression information. In response to the comparison indicating that the moisture difference is below the predetermined moisture threshold, the contralateral comparison circuit 516 determines that the wound treatment site is likely not macerated. In response to the comparison indicating that the moisture difference is above the moisture threshold, the contralateral comparison circuit 516 determines that wound treatment site is likely macerated. The contralateral comparison circuit 516 can send a notification to a physician and/or the patient indicating that the wound treatment site is likely macerated.

In some embodiments, the physiological characteristic is a pH and the first wound sensor assembly 104 and the second wound sensor assembly 108 each includes the pH sensor 144. The contralateral comparison circuit 516 receives pH data representative of the wound treatment site from the first wound sensor assembly 104 and receives pH data representative of the healthy tissue site from the second wound sensor assembly 108. The contralateral comparison circuit 516 then determines the difference between the pH of the wound treatment site and the pH of the healthy tissue. The contralateral comparison circuit 516 then compares the pH difference to a predetermined pH threshold to determine wound healing progression information. In response to the comparison indicating that the pH difference is below the predetermined pH threshold, the contralateral comparison circuit 516 determines that an inhibition to wound healing is likely not present at the wound treatment site. In response to the comparison indicating that the pH difference is above the pH threshold, the contralateral comparison circuit 516 determines that wound treatment site is likely has an inhibition to wound healing. The contralateral comparison circuit 516 can send a notification to a physician and/or the patient indicating that the wound treatment site likely has an inhibition to wound healing.

In embodiments including the powered NPWT system 112, the NPWT control circuit 520 is in communication with the pump 152 and one or more pressure sensors. In some embodiments, the wound dressing 312, 412 can include a pressure sensor. In other embodiments, the first wound sensor assembly 104 and the second wound sensor assembly 108 can each include the pressure sensor 148. The NPWT control circuit 520 is configured to determine a pressure at the wound treatment site in response to receiving pressure data representative of the wound treatment site from the pressure sensor of the wound dressing 312, 412 or the pressure sensor 148 of the first wound sensor assembly 104. The NPWT control circuit 520 is configured to control the pump 152 to provide negative pressure to the wound treatment site based on the determined pressure at the wound treatment site. For example, the NPWT control circuit 520 can increase, decrease, or maintain an amount of negative pressure provided to the wound treatment site.

In embodiments including the non-powered NPWT system 112, the NPWT control circuit 520 be configured to receive pressure data representative of the wound treatment site sensed by the pressure sensor 148 of the first wound sensor assembly or a pressure sensor of the wound dressing 312, 412. The NPWT control circuit 520 can compare the pressure data to a predetermined threshold, such as a pressure threshold established in a wound treatment regimen. In response to determining that the pressure data representative of the wound treatment site is above a predetermined threshold, the NPWT control circuit 520 can send a notification to the patient via the monitoring device 216 prompting the patient to actuate the NPWT system 112 to increase an amount of negative pressure applied to the wound treatment site.

In embodiments in which the second wound sensor assembly 108 includes the pressure sensor 148, the NPWT control circuit 520 is configured to determine a difference in the pressure data representative of the wound treatment site and the pressure data representative of the healthy tissue site. The NPWT control circuit 520 is configured to determine a difference between the pressure representative of the wound treatment site and the pressure data representative of the healthy tissue site. The NPWT control circuit 520 is configured to compare the pressure difference to a predetermined pressure threshold to determine information indicative of wound healing progression. In response to the comparison indicating that the pressure difference is below the predetermined threshold, the NPWT control circuit 520 is configured to determine that the patient is likely not complying with a NPWT treatment regimen. The NPWT control circuit 520 is configured to send a notification to a patient's caregiver and/or to the patient indicating that the patient is likely not complying with the NPWT therapy regime.

In embodiments including the instillation systems 116, 224, the instillation control circuit 524 is in communication with the instillation system 116, 224 and the moisture sensor 140. In some embodiments, the wound dressing 312, 412 can include a moisture sensor. In other embodiments, the first wound sensor assembly 104 can include the moisture sensor 140. The instillation control circuit 524 is configured to determine a moisture at the wound treatment site in response to receiving moisture data representative of the wound treatment site from the moisture sensor of the wound dressing 312, 412 or the moisture sensor of the first wound sensor assembly 104. The instillation control circuit 524 is configured to control the pump 178 to provide instillation fluid to the wound treatment site based on the determined moisture at the wound treatment site. For example, the instillation control circuit 524 can increase, decrease, or maintain an amount of instillation fluid provided to the wound treatment site based on the moisture data. The instillation control circuit 544 can also increase, decrease, or maintain a dwell time of the instillation fluid at the wound treatment site based on the moisture data.

A block diagram illustrating the controller 220 in greater detail is shown in FIG. 6, according to an exemplary embodiment. The controller 220 can be used with various wound dressings and sensors described herein. The controller 220 includes a processing circuit 604 including a processor 608 and memory 612. The processor 608 may be a general purpose or specific purpose processor, an ASIC, one or more FPGAs, a group of processing components, or other suitable processing components. The processor 608 is configured to execute computer code or instructions stored in the memory 612 or received from other computer readable media (e.g., CDROM, network storage, a remote server, etc.). The processing circuit 504 includes a contralateral comparison circuit 516. In embodiments in which the wound therapy system 100 includes the instillation system 116, the processing circuit 504 also includes an instillation control circuit 524. The controller 220 is similar to the controller 120. Accordingly, the controller 220 is not discussed in detail herein.

### Method for Determining Wound Healing Information Indicative of Wound Healing Progression

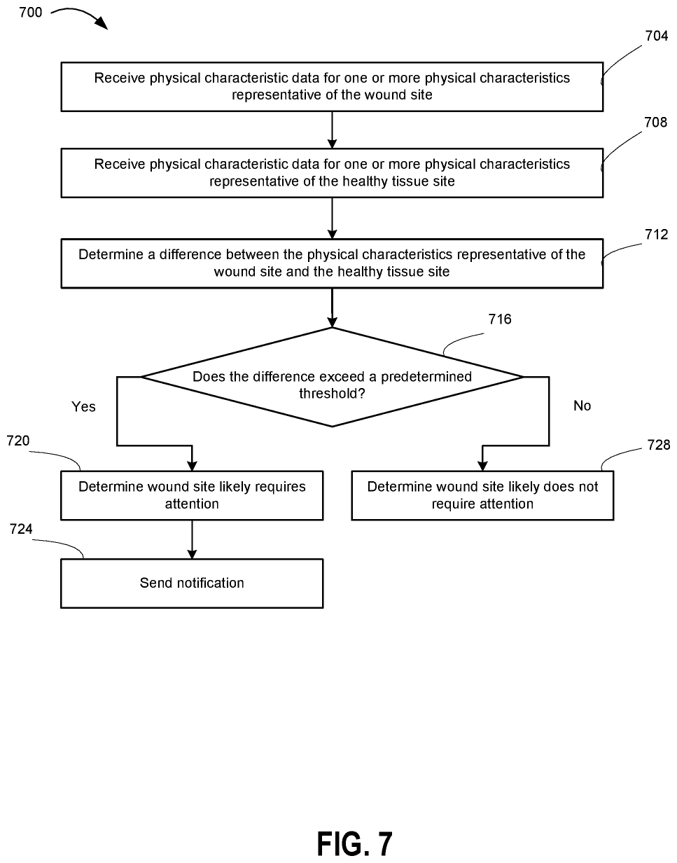

FIG. 7 illustrates a method 700 for determining wound healing information indicative of wound healing progression for a wound treatment site according to an exemplary embodiment. At step 704, the contralateral comparison circuit 516 is configured to receive physiological characteristics data for one or more physiological characteristics of the wound treatment site sensed by the first wound sensor assembly 104. For example, the first wound sensor assembly 104 can sense physiological characteristics data representative of the wound treatment site using the one or more sensors 132-148. The first wound sensor assembly 104 can transmit the sensed physiological characteristic data to the controller 120 over the communication interface 124. At step 708, the contralateral comparison circuit 516 is configured to receive physiological characteristic data representative of the healthy tissue site sensed by the second wound sensor assembly 108. For example, the second wound sensor assembly 108 can sense physiological characteristics data representative of the healthy tissue site using the one or more sensors 132-148. The second wound sensor assembly 108 can transmit the physiological characteristics data to the controller 120 over the communication interface 124.

At step 712, the contralateral comparison circuit 516 is configured to determine a difference between the physiological characteristics of the wound treatment site and the physiological characteristics of the healthy tissue site. At step 716, the contralateral comparison circuit 516 is configured to compare the difference to a predetermined threshold to determine information indicative of a wound healing progression. At step 720, the contralateral comparison circuit 516 is configured to determine that the wound healing progression likely includes one or more situations requiring caregiver attention in response to determining that the difference between the physiological characteristics of the wound treatment site and the physiological characteristics of the healthy tissue site exceeds a predetermined threshold. At step 724, the contralateral comparison circuit 516 can send a notification to the patient and/or the caregiver in response to the comparison indicating that a situation requiring caregiver attention is likely present. At step 728, the contralateral comparison circuit 516 is configured to determine that the wound treatment site healing progression likely does not include one or more situations requiring caregiver attention in response to determining that the difference between the physiological characteristics of the wound treatment site and the physiological characteristics of the healthy tissue site does not exceed the predetermined threshold.

In some embodiments the one or more physiological characteristics includes temperature. In such embodiments, the contralateral comparison circuit 516 receives temperature data representative of the wound treatment site sensed by the first wound sensor assembly 104. For example, the first wound sensor assembly 104 senses a periwound temperature representative of the wound treatment site using the temperature sensor 132. The first wound sensor assembly 104 transmits the sensed temperature data to the controller 120 over the communication interface 124. The contralateral comparison circuit 516 receives temperature data representative of the healthy tissue site sensed by the second wound sensor assembly 108. The second wound sensor assembly 108 senses a temperature representative of the healthy tissue site using the temperature sensor 132. The second wound sensor assembly 108 can transmit the temperature data to the controller 120 over the communication interface 124.

The contralateral comparison circuit 516 determines a difference between the temperature data of the wound treatment site and the temperature data of the healthy tissue site. For example, the contralateral comparison circuit 516 subtracts a value the temperature data of the wound treatment site and the temperature data of the healthy from the other of the wound treatment site and the healthy tissue site to determine the difference. In some embodiments, the contralateral comparison circuit 516 uses the absolute value of the result of the subtraction as the difference. The contralateral comparison circuit 516 compares the difference to a predetermined temperature threshold to determine information indicative of a wound healing progression. The contralateral comparison circuit 516 determines that the wound treatment site healing progression likely includes an infection in response to the difference between the temperature data of the wound treatment site and the temperature of the healthy tissue site exceeding a predetermined threshold. The contralateral comparison circuit 516 sends a notification to the patient and/or the caregiver in response to the comparison indicating that an infection is likely present. The contralateral comparison circuit 516 determines that the wound treatment site healing progression likely does not include an infection in response to difference between the temperature data of the wound treatment site and the temperature data of the healthy tissue site being below a predetermined threshold.

In embodiments in which the one or more physiological characteristics includes moisture, the contralateral comparison circuit 516 receives moisture data representative of the wound treatment site sensed by the moisture sensor 140 of the first wound sensor assembly 104. For example, the first wound sensor assembly 104 senses periwound moisture data representative of the wound treatment site using the moisture sensor 140. The first wound sensor assembly 104 transmits the sensed moisture data to the controller 120 over the communication interface 124. The contralateral comparison circuit 516 receives moisture data representative of the healthy tissue site sensed by the second wound sensor assembly 108. The second wound sensor assembly 108 senses the moisture data representative of the healthy tissue site using the moisture sensor 140. The second wound sensor assembly 108 transmits the moisture data to the controller 120 over the communication interface 124.

The contralateral comparison circuit 516 determines a difference between the moisture data of the wound treatment site and the moisture data of the healthy tissue site. The contralateral comparison circuit 516 compares the difference to a predetermined moisture threshold to determine information indicative of a wound healing progression. The contralateral comparison circuit 516 determines that the wound treatment site healing progression likely includes maceration in response to the difference between the moisture data of the wound treatment site and the moisture data of the healthy tissue site exceeding a predetermined threshold. The contralateral comparison circuit 516 sends a notification to the patient and/or the caregiver in response to the comparison indicating that maceration is likely present. The contralateral comparison circuit 516 determines that the wound treatment site healing progression likely does not include maceration in response to difference between the moisture data of the wound treatment site and the moisture data of the healthy tissue site being below a predetermined threshold.

In embodiments in which the one or more physiological characteristics includes pH, the contralateral comparison circuit 516 receives pH data representative of the wound treatment site sensed by the first wound sensor assembly 104. For example, the first wound sensor assembly 104 senses a periwound pH representative of the wound treatment site using the pH sensor 144. The first wound sensor assembly 104 transmits the sensed pH data to the controller 120 over the communication interface 124. The contralateral comparison circuit 516 receives pH data representative of the healthy tissue site sensed by the second wound sensor assembly 108. The second wound sensor assembly 108 can sense pH data representative of the healthy tissue site using the pH sensor 144. The second wound sensor assembly 108 transmits the pH data to the controller 120 over the communication interface 124.

The contralateral comparison circuit 516 determines a difference between the pH data representative of the wound treatment site and the pH data representative of the healthy tissue site. The contralateral comparison circuit 516 compares the difference to a predetermined pH threshold to determine information indicative of a wound healing progression. The contralateral comparison circuit 516 determines that the wound treatment site healing progression likely includes an inhibition to healing in response to the difference between the pH data of the wound treatment site and the pH data of the healthy tissue site exceeding a predetermined threshold. The contralateral comparison circuit 516 sends a notification to the patient and/or the caregiver in response to the comparison indicating that an inhibition to healing is likely present. The contralateral comparison circuit 516 determines that the wound treatment site healing progression likely does not include an inhibition to healing in response to difference between the pH data of the wound treatment site and the pH data of the healthy tissue site exceeding a predetermined threshold.

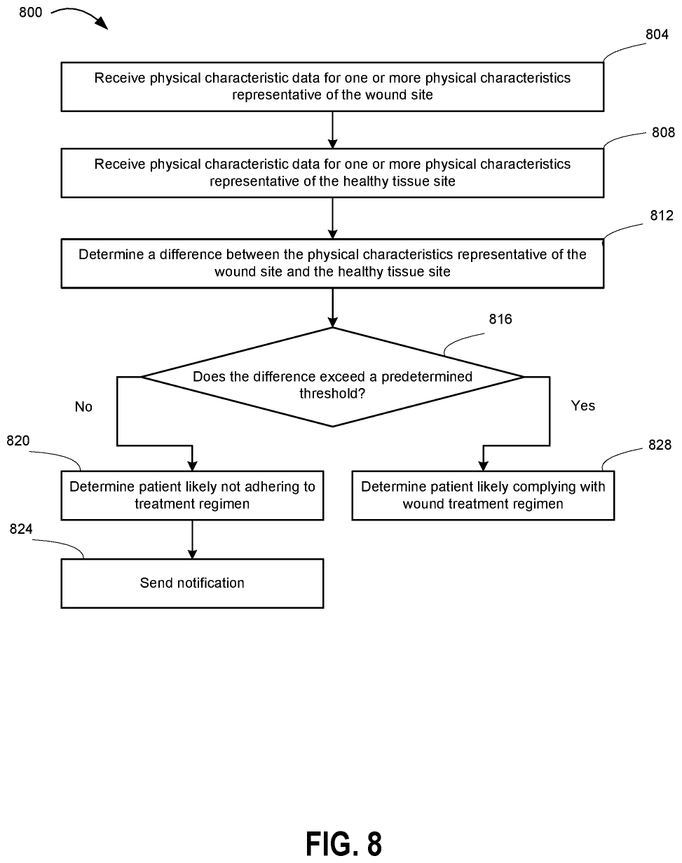

FIG. 8 illustrates a method 800 for determining patient non-compliance with a wound treatment regimen for a wound treatment site according to an exemplary embodiment. At step 804, the contralateral comparison circuit 516 is configured to receive physiological characteristics data for one or more physiological characteristics representative of the wound treatment site sensed by the first wound sensor assembly 104. For example, the first wound sensor assembly 104 can sense one or more of the physiological characteristics representative of the wound treatment site using the one or more sensors 132-148. The first wound sensor assembly 104 can transmit the sensed physiological characteristic data to the controller 120 over the communication interface 124. At step 808, the contralateral comparison circuit 516 is configured to sense physiological characteristic data representative of the physiological characteristics of the healthy tissue site sensed by the second wound sensor assembly 108 using the one or more sensors 132-148. The second wound sensor assembly 108 can transmit the physiological characteristics to the controller 120 over the communication interface 124.

At step 812, the contralateral comparison circuit 516 is configured to determine a difference between the physiological characteristics representative of the wound treatment site and the physiological characteristics representative of the healthy tissue site. At step 816, the contralateral comparison circuit 516 is configured to compare the difference to a predetermined threshold to determine information indicative of a wound healing progression. At step 820, the contralateral comparison circuit 516 is configured to determine that the patient is likely not complying with a wound healing regimen in response to the difference between the physiological characteristics of the wound treatment site and the physiological characteristics of the healthy tissue site exceeding a predetermined threshold. At step 824, the contralateral comparison circuit 516 can send a notification to the patient and/or the caregiver in response to the comparison indicating that the patient is likely not complying with the wound treatment regimen. At step 828, the contralateral comparison circuit 516 is configured to determine that the patient is likely complying with the wound treatment regimen in response to difference between the physiological characteristics representative of the wound treatment site and the physiological characteristics representative of the healthy tissue site being below a predetermined threshold.

For example, in embodiments in which the physiological characteristic includes motion, the contralateral comparison circuit 516 receives motion data representative of the wound treatment site sensed by the first wound sensor assembly 104. For example, the first wound sensor assembly senses motion data representative of the wound treatment site using the motion sensor 136. The first wound sensor assembly 104 transmits the sensed motion data to the controller 120 over the communication interface 124. The contralateral comparison circuit 516 receives motion data representative of the physiological characteristics of the healthy tissue site sensed by the second wound sensor assembly 108 using the motion sensor 136. The second wound sensor assembly 108 transmits the motion data to the controller 120 over the communication interface 124.

The contralateral comparison circuit 516 determines a difference between the motion data of the wound treatment site and the motion data of the healthy tissue site. The contralateral comparison circuit 516 compares the difference to a predetermined threshold to determine information indicative of a wound healing progression. The contralateral comparison circuit 516 determines that the patient is likely complying with a wound healing regimen in response to the difference between the physiological characteristics representative of the wound treatment site and the physiological characteristics representative of the healthy tissue site exceeding a predetermined threshold. For example, the treatment regimen can include restricting a range of motion or an amount of activity of the extremity including the wound treatment site relative to the extremity including the healthy tissue site. The contralateral comparison circuit 516 is configured to determine that the patient is likely not complying with the wound treatment regimen in response to difference between the physiological characteristics representative of the wound treatment site and the physiological characteristics representative of the healthy tissue site being less than a predetermined threshold. For example, a difference in motion data less than the predetermined threshold can indicate overuse of the extremity including the wound treatment site. The contralateral comparison circuit 516 sends a notification to the patient and/or the caregiver in response to the comparison indicating that the patient is likely not complying with the wound treatment regimen.

In embodiments in which the physiological characteristic includes pressure, the contralateral comparison circuit 516 receives pressure data representative of the wound treatment site sensed by the first wound sensor assembly 104. For example, the first wound sensor assembly senses pressure data representative of the wound treatment site using the pressure sensor 148. The first wound sensor assembly 104 transmits the sensed pressure data to the controller 120 over the communication interface 124. The contralateral comparison circuit 516 receives pressure data representative of the healthy tissue site sensed by the second wound sensor assembly 108 using the pressure sensor 148. The second wound sensor assembly 108 transmits the pressure data to the controller 120 over the communication interface 124.

The contralateral comparison circuit 516 determines a difference between the pressure data representative of the wound treatment site and the motion data representative of the healthy tissue site. The contralateral comparison circuit 516 compares the difference to a predetermined threshold to determine information indicative of a wound healing progression. The contralateral comparison circuit 516 determines that the patient is likely complying with a wound healing regimen in response to the difference between the pressure data representative of the wound treatment site and the pressure data representative of the healthy tissue site exceeding a predetermined threshold. During NPWT treatment, the wound treatment site is expected to have a lower pressure than the healthy tissue site since the healthy tissue site is not subjected to negative pressure. The contralateral comparison circuit 516 determines that the patient is likely not complying with the wound treatment regimen in response to difference between the pressure representative of the wound treatment site and the pressure representative of the healthy tissue site falling below a predetermined threshold. For example, a difference in pressure data less than the predetermined threshold can indicate that a patient is not applying negative pressure to the wound treatment site. The contralateral comparison circuit 516 sends a notification to the patient and/or the caregiver in response to the comparison indicating that the patient is likely not complying with the wound treatment regimen. In some embodiments, the notification sent to the patient can prompt the patient to increase an amount of negative pressure applied to the wound treatment site.

### Configuration of Exemplary Embodiments

The construction and arrangement of the systems and methods as shown in the various exemplary embodiments are illustrative only. Although only a few embodiments have been described in detail in this disclosure, many modifications are possible (e.g., variations in sizes, dimensions, structures, shapes and proportions of the various elements, values of parameters, mounting arrangements, use of materials, colors, orientations, etc.). For example, the position of elements can be reversed or otherwise varied, and the nature or number of discrete elements or positions can be altered or varied. Accordingly, all such modifications are intended to be included within the scope of the present disclosure. The order or sequence of any process or method steps can be varied or re-sequenced according to alternative embodiments. Other substitutions, modifications, changes, and omissions can be made in the design, operating conditions and arrangement of the exemplary embodiments without departing from the scope of the present disclosure.

1. A wound therapy system comprising:
a first sensor assembly configured to be located at a wound treatment site on a patient's body and to record one or more physical characteristics representative of periwound tissue at the wound treatment site;
a second sensor assembly configured to be located at a healthy tissue site on the patient's body contralateral to the wound treatment site and to record one or more physical characteristics representative of healthy tissue at the healthy tissue site, the physical characteristics representative of the healthy tissue recorded concurrently with the physical characteristics representative of the periwound tissue; and
a processing circuit configured to:
receive the one or more physical characteristics representative of the periwound tissue from the first sensor assembly at the wound treatment site;
receive the one or more physical characteristics representative of the healthy tissue from the second sensor assembly at the healthy tissue site; and
determine a healing progression of the wound based on a difference between the one or more physical characteristics representative of the periwound tissue and the one or more physical characteristics representative of the healthy tissue.

2. The wound therapy system of claim 1, wherein the wound treatment site is located on a first extremity of the patient's body and the healthy tissue site is located on a second extremity of the patient's body contralateral to the first extremity.

3. (canceled)

4. The wound therapy system of claim 1, wherein:
the first sensor assembly comprises a first communications interface configured to transmit the one or more physical characteristics representative of the periwound tissue to the processing circuit; and
the second sensor assembly comprises a second communications interface configured to transmit the one or more physical characteristics representative of the healthy tissue to the processing circuit.

5. The wound therapy system of claim 4, wherein first communications interface and the second communications interface are wireless communications interfaces configured to transmit the one or more physical characteristics representative of the periwound tissue and the one or more physical characteristics representative of the healthy tissue using a wireless communications protocol.

6. The wound therapy system of claim 5, wherein the wireless communications protocol comprises at least one of Bluetooth, cellular, or Wi-Fi.

7. The wound therapy system of claim 1, further comprising a negative pressure wound therapy (NPWT) device configured to apply negative pressure to the wound treatment site.

8. The wound therapy system of claim 7, wherein the NPWT system is a powered device comprising the processing circuit.

9. The wound therapy system of claim 7, wherein the NPWT system is a non-powered device and the processing circuit is a component of a separate monitoring device.

10. (canceled)

11. The wound therapy system of claim 1, wherein:
the first sensor assembly comprises a first temperature sensor configured to measure a first temperature of the periwound tissue;
the second sensor assembly comprises a second temperature sensor configured to measure a second temperature of the healthy tissue; and
the processing circuit is configured to detect an infection of the wound in response to a difference between the first temperature and the second temperature exceeding a threshold temperature difference.

12. The wound therapy system of claim 1, wherein:
the first sensor assembly comprises a first moisture sensor configured to measure a first moisture of the periwound tissue;
the second sensor assembly comprises a second moisture sensor configured to measure a second moisture of the healthy tissue; and
the processing circuit is configured to detect a maceration of the wound in response to a difference between the first moisture and the second moisture exceeding a threshold moisture difference.

13. The wound therapy system of claim 1, wherein:
the first sensor assembly comprises a first motion sensor configured to measure a first motion of the periwound tissue;
the second sensor assembly comprises a second motion sensor configured to measure a second motion of the healthy tissue; and
the processing circuit is configured to detect overuse of an injured appendage or patient non-compliance with restricted movement instructions based on at least one of the first motion or the second motion.

14. The wound therapy system of claim 1, wherein:
the first sensor assembly comprises a first motion sensor configured to measure a first motion of the periwound tissue;
the second sensor assembly comprises a second motion sensor configured to measure a second motion of the healthy tissue; and
the processing circuit is configured to detect improvement in patient mobility in response to a difference between the first movement and the second movement dropping below a threshold movement difference.

15. The wound therapy system of claim 1, wherein:
the first sensor assembly comprises a first pH sensor configured to measure a first pH of the periwound tissue;
the second sensor assembly comprises a second pH sensor configured to measure a second pH of the healthy tissue; and
the processing circuit is configured to detect an inhibition to wound healing in response to a difference between the first pH and the second pH exceeding a threshold pH difference.

16. The wound therapy system of claim 1, wherein:
the first sensor assembly comprises a first pressure sensor configured to measure a first pressure of the periwound tissue;
the second sensor assembly comprises a second pressure sensor configured to measure a second pressure of the healthy tissue; and
the processing circuit is configured to detect insufficient negative pressure at the wound treatment site in response to a difference between the first pressure and the second pressure being less than a threshold pressure difference.

17. (canceled)

18. The wound therapy system of claim 1, further comprising a fluid instillation device configured to deliver instillation fluid to the wound treatment site;
wherein the processing circuit is configured to:
calculate a difference between the one or more physical characteristics representative recorded by the first sensor assembly and the one or more physical characteristics representative recorded by the second sensor assembly; and
cause the fluid instillation device to deliver the instillation fluid to the wound treatment site in response to the calculated difference exceeding a threshold difference.

19. A method for monitoring wound healing, the method comprising:
recording one or more physical characteristics representative of periwound tissue at a wound treatment site on a patient's body using a first sensor assembly located at the wound treatment site;
recording one or more physical characteristics representative of healthy tissue at a healthy tissue site on the patient's body contralateral to the wound treatment site using a second sensor assembly located at a healthy tissue site, the physical characteristics representative of the healthy tissue recorded concurrently with the physical characteristics representative of the periwound tissue;
transmitting the one or more physical characteristics representative of the periwound tissue from the first sensor assembly to a processing circuit;
transmitting the one or more physical characteristics representative of the healthy tissue from the second sensor assembly to the processing circuit; and
determining, by the processing circuit, a healing progression of the wound based on a difference between the one or more physical characteristics representative of the periwound tissue and the one or more physical characteristics representative of the healthy tissue.

20. (canceled)

21. The method of claim 19, wherein the healing progression is determined by a monitoring device separate from the first sensor assembly and the second sensor assembly.

22. The method of claim 21, comprising:
transmitting the one or more physical characteristics representative of the periwound tissue from the first sensor assembly to the monitoring device via a first communications interface of the first sensor assembly; and
transmitting the one or more physical characteristics representative of the healthy tissue from the second sensor assembly to the monitoring device via a second communications interface of the second sensor assembly.

23. (canceled)

24. (canceled)

25. The method of claim 19, further comprising operating a negative pressure wound therapy (NPWT) device to apply negative pressure to the wound treatment site.

26. The method of claim 25, wherein the NPWT system is a powered device and the healing progression is determined by the NPWT system.

27. (canceled)

28. The method of claim 19, further comprising powering at least one of the first sensor assembly or the second sensor assembly using a battery of the first sensor assembly or the second sensor assembly.

29. The method of claim 19, wherein:
the one or more physical characteristics representative of the periwound tissue comprise a first temperature recorded by a first temperature sensor of the first sensor assembly;
the one or more physical characteristics representative of the healthy tissue comprise a second temperature recorded by a second temperature sensor of the second sensor assembly; and
the method comprises detecting an infection of the wound in response to a difference between the first temperature and the second temperature exceeding a threshold temperature difference.

30. The method of claim 19, wherein:
the one or more physical characteristics representative of the periwound tissue comprise a first moisture recorded by a first moisture sensor of the first sensor assembly;
the one or more physical characteristics representative of the healthy tissue comprise a second moisture recorded by a second moisture sensor of the second sensor assembly; and
the method comprises detecting a maceration of the wound in response to a difference between the first moisture and the second moisture exceeding a threshold moisture difference.

31. The method of claim 19, wherein:
the one or more physical characteristics representative of the periwound tissue comprise a first motion recorded by a first motion sensor of the first sensor assembly;
the one or more physical characteristics representative of the healthy tissue comprise a second motion recorded by a second motion sensor of the second sensor assembly; and
the method comprises detecting overuse of an injured appendage or patient non-compliance with restricted movement instructions based on at least one of the first motion or the second motion.

32. The method of claim 19, wherein:
the one or more physical characteristics representative of the periwound tissue comprise a first motion recorded by a first motion sensor of the first sensor assembly;
the one or more physical characteristics representative of the healthy tissue comprise a second motion recorded by a second motion sensor of the second sensor assembly; and
the method comprises detecting improvement in patient mobility in response to a difference between the first movement and the second movement dropping below a threshold movement difference.

33. The method of claim 19, wherein:
the one or more physical characteristics representative of the periwound tissue comprise a first pH recorded by a first pH sensor of the first sensor assembly;
the one or more physical characteristics representative of the healthy tissue comprise a second pH recorded by a second pH sensor of the second sensor assembly; and
the method comprises detecting an inhibition to wound healing in response to a difference between the first pH and the second pH exceeding a threshold pH difference.

34. The method of claim 19, wherein:
the one or more physical characteristics representative of the periwound tissue comprise a first pressure recorded by a first pressure sensor of the first sensor assembly;
the one or more physical characteristics representative of the healthy tissue comprise a second pressure recorded by a second pressure sensor of the second sensor assembly; and
the method comprises detecting insufficient negative pressure at the wound treatment site in response to a difference between the first pressure and the second pressure being less than a threshold pressure difference.

35. (canceled)

36. The method of claim 19, further comprising:
calculating a difference between the one or more physical characteristics representative recorded by the first sensor assembly and the one or more physical characteristics representative recorded by the second sensor assembly; and
operating a fluid instillation device to deliver instillation fluid to the wound treatment site in response to the calculated difference exceeding a threshold difference.
